(19) United States
(12) Patent Application Publication
(10) Pub. No.: US 2023/0381135 A1
(43) Pub. Date: Nov. 30, 2023

(54) COMPOSITION COMPRISING AT LEAST ONE LABDANE DITERPENE AND METHOD FOR MANUFACTURING SAME

(71) Applicants: TILMAN SA, Baillonville (BE); ELEONOR SPRL, Waterloo (BE)

(72) Inventors: Mayéli PRIAN, Rendeux (BE); Cristina LOIRA-PASTORIZA, Bruxelles (BE); Fabian PRIEM, Waterloo (BE); Yvan DIERCKXSENS, Overijse (BE)

(21) Appl. No.: 18/031,576

(22) PCT Filed: Oct. 13, 2021
(86) PCT No.: PCT/EP2021/078288

§ 371 Date: Apr. 12, 2023

(30) Foreign Application Priority Data
Oct. 13, 2020 (BE) 2020/5710

(51) Int. Cl. A61K 31/365; A61K 31/352; A61K 31/045; A61K 31/341; A61K 31/19; A61K 47/18; A61K 47/40; A61K 47/42; A61K 47/26; A61K 47/10; A61K 47/36; A61K 36/19

(52) U.S. Cl. CPC A61K 31/365; A61K 31/352; A61K 31/045; A61K 31/341; A61K 31/19; A61K 47/183; A61K 47/40; A61K 47/42; A61K 47/26; A61K 47/10; A61K 47/36; A61K 36/19

(57) ABSTRACT

The present invention relates to a composition in the form of a thermoformed extrudate comprising at least one labdane diterpene (also called labdane), and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

### CROSS-REFERENCE TO RELATED APPLICATIONS

This application is a National Phase Patent Application and claims priority to and the benefit of International Patent Application No. PCT/EP2021/078288, filed on Oct. 13, 2021, which claims priority to and the benefit of Belgian Patent Application No. 2020/5710, filed on Oct. 13, 2020. The entire contents of both of which are incorporated herein by reference.

The present invention relates to a composition comprising at least one labdane diterpene (also called labdane), to its manufacturing method and to its use.

In the sense of the present invention, the terms “labdane diterpene” or “labdane” mean the same compounds/molecules, namely both natural origin labdane diterpenes or labdanes (for example, from plants, algae, sponges or insects), and synthetic labdane diterpenes or labdanes, but also all derivatives of labdane diterpenes or labdanes, like for example labdane diterpene or labdane salts, or also glycosyl forms of labdane diterpenes or labdanes.

Labdane diterpenes (or labdanes) are derivatives of (C20) diterpenes, in particular bicyclic diterpenes with or without lactone function(s) and are mainly plant origin organic substances having well-known and widely used therapeutic properties (anti-inflammatory, antibacterial, antiviral properties, etc.).

Among labdane diterpenes (or labdanes) are found, for example, andrographolide, neoandrographolide, deoxyandrographolide, dehydroandrographolide, isoandrographolide, marrubiin, marrubenol, solicanolide, forskolin, galanolactone, medigenine, coronarines, andalusol, hispanolone, copalic acid, hedychenone, lagopsines, macranthine A, labdanediol, serralabdanes, leopersine, cis-communic acid or also sclareol.

It is noted that andrographolide has a particular interest for treating respiratory infections (colds, laryngitis, etc.) and fever.

Labdane diterpenes or labdanes are compounds which are very hardly even totally insoluble in water, these compounds subsequently have very low bioavailabilities. However, despite their low bioavailability/bioaccessibility (i.e. despite the small fraction of the dose administered which actually reaches the blood flow in unchanged form), but also despite their low solubility, in particular in the intestinal medium, the positive effects of labdane diterpenes (or labdanes) on various pathologies are molecules of interest in view of an administration in humans and/or for veterinary use.

That is why, numerous methodologies and numerous methods have been developed in order to formulate these compounds in the form of spherical particles, flakes, pellets or also granules. In particular, extrusion techniques are currently used like, for example the extrusion/spheronisation technique or the Tween screw granulation (TSG) technique.

The extrusion/spheronisation technique makes it possible to produce round particles of homogeneous size at the start of a humid mass (containing an active substance and at least one excipient) which is passed through a grille having a predetermined mesh before drying the particles thus obtained. More specifically, this powder shaping method is based on the following steps: mixture of an active substance and of at least one excipient, humid granulation (compaction) of the mixture previously obtained, extrusion of the compacted mixture to obtain an extrudate, spheronisation of the extrudate to form spherical particles/granules and drying of the spherical particles/granules obtained.

The Tween screw granulation technique itself makes it possible to obtain intermediate products for the preparation of tablets and capsules. This technique is based on a granulation (compaction) of substances in the form of powders in an extruder to lead to the formation of granules exiting the latter.

Unfortunately, it appears that the current formulations comprising labdane diterpenes (or labdanes), for example andrographolide, are hardly suitable due to their too low even their non-solubility in aqueous phase and/or due to the low salting out of these compounds at the start of these formulations, which is finally conveyed by a low bioavailability/bioaccessibility of these molecules of interest.

It is noted that it also appears that the current methods for manufacturing these formulations are restrictive and difficult to implement. In this sense, methods for which the implementation is restrictive exist to form labdane diterpene-cyclodextrin complexes. If the product obtained in the form of complex has an increased solubility and bioavailability with respect to non-complexed labdane diterpene, it remains no less than the methods for obtaining such complexes being particularly restrictive and that they involve the implementation of organic solvents. Indeed, to obtain a labdane diterpene-cyclodextrin complex, each of these compounds must be solubilised beforehand in a suitable and specific solvent (organic solvent for labdane diterpene; water and/or buffer for cyclodextrin) before removing the latter by evaporation and/or filtration before drying the complex to obtain a complex in the form of a powder.

By the term, “dispersion in aqueous phase (in aqueous medium”, this means, in the sense of the present invention, a system composed of two phases, wherein one of the two phases, called dispersed phase, is finely split into the other, called dispersing phase. This dispersion can be molecular (solution), colloidal (dispersion of submicronic particles) or coarser (dispersion of particles greater than 1 μm). More specifically, according to the invention, the term “dispersion in aqueous phase” means suspensions, constituted of a solid phase dispersed in an aqueous phase (liquid).

In the sense of the present invention, the term “solubility”, means the capacity of a substance, called solution, to be dissolved in another substance, called solvent, to form a homogeneous mixture called solution.

The invention aims to overcome the disadvantages of the state of the art by providing (1) a composition comprising at least one labdane diterpene (or labdane), for example andrographolide, the solubility in aqueous phase (aqueous medium) of which is increased such that the bioavailability of this compound is significantly increased, and (2) a method for manufacturing such a composition which is easy to implement, which is flexible, which is economically profitable and which ensures that said at least one labdane diterpene (or labdane), for example andrographolide, is present and distributed homogeneously in the final composition obtained.

Moreover, the invention intends to provide a composition which is stable over time, i.e. which preserves its properties in terms of solubility of said at least one labdane diterpene (or labdane), for example andrographolide, and which preserves its properties in terms of salting out rate of this compound over time at the start of a composition (formulation) according to the invention, this in particular, in aqueous phase and more specifically, in the intestinal medium.

Furthermore, and preferably, the invention intends to provide a composition, of which the different constituents/compounds are of natural origin or are natural, in particular concerning the polymer.

Preferably, according to the invention, said polymer is of natural origin.

More preferably also, according to the invention, said polymer is natural.

To resolve at least partially these problems, a composition in the form of a thermoformed extrudate comprising at least one labdane diterpene (or labdane) and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof is provided according to the invention.

It has been determined, in the scope of the present invention, that such a composition in the form of a thermoformed extrudate comprising at least one labdane diterpene (or labdane) as principal agent and at least polymer selected from the group made up of amino acids, natural or synthetic peptides and polypeptides, natural or synthetic proteins, natural or synthetic oligosaccharides, natural or synthetic non-cellulose polysaccharides, derivates thereof and mixtures thereof has a solubility in aqueous phase (aqueous medium) clearly greater than said at least one labdane diterpene (or labdane).

Moreover, it has been shown that such a composition according to the invention has a significantly increased bioavailability/bioaccessibility of said at least one labdane diterpene (or labdane) with respect to the bioavailabilities/bioaccessibilities observed for the current compositions comprising this compound.

Furthermore, a composition according to the invention can be preserved for several months without its properties being altered. In particular, it has been highlighted that a composition according to the invention preserves its properties in terms of solubility in aqueous phase (aqueous medium) of said at least one labdane diterpene (or labdane) and in terms of salting out rate of this compound over time at the start of a composition/formulation according to the invention, this in particular, in aqueous phase.

According to the invention, the principal agent, i.e. said at least one labdane diterpene (or labdane) is dispersed/distributed homogeneously within at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, the principal agent and/or said at last one polymer being melted during the manufacturing method by thermoforming implemented according to the invention and described below.

In particular, according to an embodiment according to the invention, a composition in the form of a thermoformed extrudate comprising at least one labdane diterpene (or labdane) and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivative thereof and mixtures thereof is provided.

In particular, according to another embodiment according to the invention, a composition in the form of a thermoformed extrudate comprising andrographolide and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivative thereof and mixtures thereof is provided.

In particular, according to another embodiment according to the invention, a composition in the form of a thermoformed extrudate comprising forskolin and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivative thereof and mixtures thereof is provided.

In particular, according to the invention, said at least one polymer is a thermoplastic polymer, i.e. a polymer having the property of softening/melting when it is heated sufficiently, but which, when cooled, becomes hard.

By the term “extrudate”, this means, in the sense of the present invention, a material which exits from an extruder, in particular from the die of an extruder.

By the term “thermoformed extrudate”, this means, in the sense of the present invention, a material which exits from equipment, in particular which exits from an extruder, wherein it has undergone a transformation by the effect of heat, possibly by the combined effect of heat and shearing forces of a worm screw. Such a transformation by the effect of heat, possibly by the combined effect of heat and shearing forces of a worm screw, can be obtained with the hot melt extrusion (HME) technique.

According to the invention, hot melt extrusion means that the agent (labdane diterpene) and the medium (polymer) are (preferably simultaneously) mixed, heated, melted, homogenised and extruded. An intense mixture and a forced stirring, in particular by worm screws during the hot melt process causes the disaggregation of agent particles in the molten polymer leading to a solid dispersion. By definition, the hot melt extrusion transforms a solid mass into a viscous liquid or into a semi-solid mass by the action of the heat of the mixture. The material (agent +polymer) is found in a “glassy state” or “glassy structure” and behaves like a brittle solid, but without a crystalline structure and having weak arrangement.

Advantageously, according to the invention, said at least one labdane diterpene (or labdane) is present at a rate of 10% to 60% by weight with respect to the total weight of the composition, preferably at a rate of 15% to 50% by weight with respect to the total weight of the composition, preferably at a rate of 20% to 40% by weight with respect to the total weight of the composition.

Preferably, according to the invention, said at least one labdane diterpene (or labdane) is present at a rate of 20% by weight with respect to the total weight of the composition, or at a rate of 30% by weight with respect to the total weight of the composition, or at a rate of 35% by weight with respect to the total weight of the composition, or at a rate of 40% by weight with respect to the total weight of the composition, or at a rate of 50% by weight with respect to the total weight of the composition, or at a rate of 60% by weight with respect to the total weight of the composition.

Preferably, according to the invention, said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivative thereof and mixtures thereof, is present at a rate of 20% to 80% by weight with respect to the total weight of the composition, preferably at a rate of 25% to 65% by weight with respect to the total weight of the composition, preferably at a rate of 30% to 60% by weight with respect to the total weight of the composition, more preferably at a rate of 40% to 55% by weight with respect to the total weight of the composition.

Advantageously, according to the invention, said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivative thereof and mixtures thereof, is present at a rate of 20% by weight with respect to the total weight of the composition, or at a rate of 25% by weight with respect to the total weight of the composition, or at a rate of 30% by weight with respect to the total weight of the composition, or at a rate of 35% by weight with respect to the total weight of the composition, or at a rate of 40% by weight with respect to the total weight of the composition, or at a rate of 45% by weight with respect to the total weight of the composition, or at a rate of 50% by weight with respect to the total weight of the composition, or at a rate of 55% by weight with respect to the total weight of the composition, or at a rate of 60% by weight with respect to the total weight of the composition, or at a rate of 65% by weight with respect to the total weight of the composition.

When this is a mixture of two polymers according to the invention, the ratio between these two polymers is preferably around 10:90 or around 20:80 or around 30:70 or around 40:60 or around 50:50.

Preferably, according to the invention, said thermoformed extrudate consists of a solid dispersion.

Advantageously, according to the invention, said solid dispersion consists of a glassy structure comprising a molecular mixture of said at least one labdane diterpene (or labdane) and of said at least one polymer.

In particular, a thermoformed extrudate according to the invention is an extrudate, wherein said at least one labdane diterpene (or labdane) and/or said at least one polymer has/have been melted to lead to a solid dispersion consisting of a glassy structure comprising a molecular mixture of said at least one labdane diterpene (or labdane) and of said at least one polymer such that said at least one labdane diterpene (or labdane) is dispersed within said at least one polymer such that said at least one labdane diterpene (or labdane) is dispersed within said at least one polymer.

In particular, a thermoformed extrudate according to the invention is an extrudate wherein said at least one labdane diterpene (or labdane) and/or said at least one polymer has/have been melted to lead to a solid dispersion consisting of a glassy structure comprising a molecular mixture of said at least one labdane diterpene (or labdane) and of said at least one polymer, such that said at least one labdane diterpene (or labdane) is dispersed within said at least one polymer.

In particular, a thermoformed extrudate according to the invention is an extrudate wherein andrographolide and/or said at least one polymer has/have been melted to lead to a solid dispersion consisting of a glassy structure comprising a molecular mixture of said andrographolide and or said at least one polymer, such that said andrographolide is dispersed within said at least one polymer.

In particular, a thermoformed extrudate according to the invention is an extrudate wherein forskolin and/or said at least one polymer has/have been melted to lead to a solid dispersion consisting of a glassy structure comprising a molecular mixture of said forskolin and or said at least one polymer, such that said forskolin is dispersed within said at least one polymer.

By the term “glassy structure”, this means, in the sense of the present invention, a structure comprising/being formed by a molecular mixture of at least two compounds/molecules, at least one of these two compounds/molecules being presented at least partially in a non-crystalline form, in particular being presented at least partially in amorphous form. In particular, in the scope of the present invention, such a glassy structure is obtained by a change of state of the material comprising the principal agent (labdane diterpene) and/or the polymer implemented, which change of state of the material is obtained by hot melt extrusion, the material being subjected to a heating and undergoing melting.

By the term “solid dispersion”, this means, in the sense of the present invention, a mixture/a system of at least two compounds/molecules, at least one of these two compounds/molecules being presented at least partially in a non-crystalline form, in particular being presented at least partially in amorphous form.

In particular, a “solid dispersion” consists of a molecular dispersion of a principal agent/an active substance at least partially in amorphous form within a polymeric matrix.

In particular, the composition according to the invention, more specifically the composition in the form of a thermoformed extrudate according to the invention is a solid dispersion, in particular a solid dispersion consisting of a glassy structure, wherein said at least one labdane diterpene (or labdane) is dispersed in said at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

According to an embodiment, a composition in the form of a thermoformed extrudate comprising at least one labdane diterpene (or labdane) and at least one amino acid, one peptides or one natural or synthetic polypeptide, as a polymer, is provided according to the invention, said composition being a solid dispersion, in particular a solid dispersion consisting of a glassy structure, wherein said at least one labdane diterpene (or labdane) is dispersed in said at least one amino acid, one peptide or one natural or synthetic polypeptide, as a polymer, which is/has been melted.

According to an embodiment, a composition in the form of a thermoformed extrudate comprising at least one labdane diterpene (or labdane) and at least one natural or synthetic protein, as a polymer, is provided according to the invention, said composition being a solid dispersion, in particular a solid dispersion consisting of a glassy structure, wherein said at least one labdane diterpene (or labdane) is dispersed in said at least one natural or synthetic protein, as a polymer, which is/has been melted.

According to another embodiment, a composition in the form of a thermoformed extrudate comprising at least one labdane diterpene (or labdane) and at least one natural or synthetic oligosaccharide, is provided according to the invention, said composition being a solid dispersion, in particular a solid dispersion consisting of a glassy structure, wherein said at least one labdane diterpene (or labdane) is dispersed in said at least one natural or synthetic oligosaccharide, as a polymer, which is/has been melted.

According also to another embodiment, a composition in the form of a thermoformed extrudate comprising at least one labdane diterpene (or labdane) and at least one natural or synthetic polysaccharide, is provided according to the invention, said composition being a solid dispersion, in particular a solid dispersion consisting of a glassy structure, wherein said at least one labdane diterpene (or labdane) is dispersed in said at least one natural or synthetic polysaccharide, as a polymer, which is/has been melted.

A thermoformed extrudate according to the invention is obtained by hot melt thermoforming, in particular by hot melt thermoforming by the hot melt extrusion technique. According to the invention, the hot melt thermoforming therefore relates more specifically to the hot melt extrusion technique.

It is therefore provided, according to the invention, a composition obtained by hot melt thermoforming in the form of a thermoformed extrudate obtained by hot melt thermoforming, in particular obtained by hot melt extrusion, said composition comprising at least one labdane diterpene (or labdane) and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

In particular, a composition obtained by hot melt thermoforming in the form of a thermoformed extrudate obtained by hot melt thermoforming, in particular obtained by hot melt extrusion is provided according to the invention, said composition comprising andrographolide and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

In particular, a composition obtained by hot melt thermoforming in the form of a thermoformed extrudate obtained by hot melt thermoforming, in particular obtained by hot melt extrusion is provided according to the invention, said composition comprising forskolin and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

A thermoformed extrudate according to the invention can therefore be obtained according to a hot melt extrusion technique which makes it possible to achieve the molecular dispersion of an agent (active substance) within a polymeric matrix (within a polymer) to form solid dispersions, the agent and/or the polymer implemented undergoing a change of state of the material. The agent and/or the polymer therefore passes from a first state of the material to a second state of the material, which second state of the material implying that said agent and/or said polymer is presented at least partially in amorphous form. This solid dispersion and this change of state of the material are possible, thanks to an input of heat and possibly thanks to the constraint applied by the movement of the worm screws on the material in an extruder. Finally, the hot melt extrusion leads to, upon exit, the formation of a thermoformed extrudate in the form of a ring which can thus in particular be pelletised or crushed.

If the extrusion-spheronisation technique and the Tween screw granulation technique such as described above are carried out without input of heat (heating) and that they typically require a liquid phase (generally water) to obtain spherical particles and/or granules without modifying the structure of the compounds/molecules implemented, the hot melt extrusion technique can be carried out without input of this liquid phase, but is based on an input of heat (heating) to ensure a transformation of the material (change of state of the material) by thermoforming, i.e. a modification of the structure of at least one of the compounds/molecules implemented, in particular an at least partial transformation of the crystalline state to the amorphous state.

Moreover, if the extrusion-spheronisation technique and the Tween screw granulation technique consist of an agglomeration of powder granules by attempting as much as possible to preserve the initial properties of the constituents of these powders, the hot melt extrusion technique, on the contrary, leads to a transformation of the material, in particular to a glass structure obtained under the action of heat (heating), the particles constituting the powders no longer being all present in their initial crystalline form (native) at the end of the hot melt extrusion method, but having undergone a transformation by thermoforming and being at least partially in amorphous form.

Preferably, according to the invention, said labdane diterpene (or labdane) is selected from the group made up of andrographolide, neoandrographolide, deoxyandrographolide, dehydroandrographolide, isoandrographolide, forskolin, marrubiin, solicanolide, sclareol, marrubenol, galanolactone, medigenine, coronarines, andalusol, hispanolone, copalic acid, hedychenone, lagopsines, macranthine A, labdanediol, serralabdanes, leopersine, cis-communic acid, derivatives thereof and mixtures thereof.

Preferably, according to the invention, said natural or synthetic proteins are selected from the group made up of glycoproteins, collagens and/or collagen hydrolysates, plant proteins, animal proteins, derivatives thereof and mixtures thereof.

As an example, among proteins, glycoproteins, collagens, collagen hydrolysates, gelatines, pea proteins, soya proteins, wheat proteins, pumpkins proteins, walnut proteins, rice proteins, derivatives thereof and mixtures thereof can therefore be mentioned.

By the term “collagen hydrolysate”, this means, in the sense of the present invention, gelatines and hydrolysed collagens or collagen peptides. The term “collagen hydrolysate” therefore include gelatines having the capacity to gelify, but also hydrolysed collagens or collagen peptides which have, or not, the capacity to gelify.

As an example, when said at least one natural or synthetic protein is collagen or gelatine, this can be collagen or gelatine of animal origin (fish, pork, beef, etc.).

As an example, when said at least one natural or synthetic protein is a plant protein, this can be a soya, pumpkin, rice, wheat, pea or walnut protein. This list is non-exhaustive.

Preferably, according to the invention, said collagens and/or said collagen hydrolysates have a molecular weight of between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

Advantageously, according to the invention, said collagens and/or said collagen hydrolysates have a molecular weight of between 1000 and 300000 Da, preferably between 1500 and 150000 Da, preferably between 2000 and 60000 Da.

Preferably, according to the invention, said collagens and/or said collagen hydrolysates have a molecular weight equal to 50 Da or equal to 100 Da or equal to 150 Da or equal to 200 Da or equal to 250 Da or equal to 300 Da or equal to 350 Da or equal to 400 Da or equal to 450 Da or equal to 500 Da or equal to 550 Da or equal to 600 Da or equal to 650 Da or equal to 700 Da or equal to 750 Da or equal to 800 Da or equal to 850 Da or equal to 900 Da or equal to 950 Da or equal to 1000 Da or equal to 1100 Da or equal to 1200 Da or equal to 1300 Da or equal to 1400 Da or equal to 1500 Da or equal to 1600 Da or equal to 1700 Da or equal to 1800 Da or equal to 1900 Da or equal to 2000 Da or equal to 2500 Da or equal to 3000 Da or equal to 3500 Da or equal to 4000 Da or equal to 4500 Da or equal to 5000 Da or equal to 5500 Da or equal to 6000 Da or equal to 6500 Da or equal to 7000 Da or equal to 7500 Da or equal to 8000 Da or equal to 8500 Da or equal to 9000 Da or equal to 9500 Da or equal to 10000 Da or equal to 12500 Da or equal to 15000 Da or equal to 17500 Da or equal to 20000 Da or equal to 22500 Da or equal to 25000 Da or equal to 27500 Da or equal to 30000 Da or equal to 32500 Da or equal to 35000 Da or equal to 37500 Da or equal to 40000 Da or equal to 42500 Da or equal to 45000 Da or equal to 47500 Da or equal to 50000 Da or equal to 55000 Da or equal to 60000 Da or equal to 65000 Da or equal to 70000 Da or equal to 75000 Da or equal to 80000 Da or equal to 85000 Da or equal to 90000 Da or equal to 100000 Da or equal to 110000 Da or equal to 120000 Da or equal to 130000 Da or equal to 140000 Da or equal to 150000 Da or equal to 160000 Da or equal to 170000 Da or equal to 180000 Da or equal to 190000 Da or equal to 200000 Da or equal to 210000 Da or equal to 220000 Da or equal to 230000 Da or equal to 240000 Da or equal to 250000 Da or equal to 260000 Da or equal to 270000 Da or equal to 280000 Da or equal to 290000 Da or equal to 300000 Da.

According to an embodiment according to the invention, when said at least one natural or synthetic protein is collagen, the latter has a molecular weight of between 900 and 7000 Da, preferably a molecular weight of between 900 and 7000 Da, preferably a molecular weight of between 950 and 5000 Da, preferably a molecular weight of between 1000 and 3000 Da.

According to an embodiment according to the invention, when said at least one natural or synthetic protein is collagen, the latter has a molecular weight equal to 2000 Da or equal to 3000 Da or equal to 5000 Da or equal to 50000 Da.

According to an embodiment according to the invention, when said at least one

natural or synthetic protein is gelatine, the latter has a molecular weight of between 900 and 6000 Da, preferably a molecular weight of between 950 and 5000 Da, preferably a molecular weight of between 1000 and 3000 Da.

According to an embodiment according to the invention, when said at least one natural or synthetic protein is gelatine, the latter has a molecular weight equal to 2000 Da or equal to 3000 Da or equal to 5000 Da or equal to 50000 Da.

According to an embodiment according to the invention, when said at least one natural or synthetic protein is a hydrolysed collagen or a collagen peptide, the latter has a molecular weight of between 900 and 6000 Da, preferably a molecular weight of between 950 and 5000 Da, preferably a molecular weight of between 1000 and 3000 Da.

According to an embodiment according to the invention, when said at least one natural or synthetic protein is a hydrolysed collagen or a collagen peptide, the latter has a molecular weight equal to 2000 Da or equal to 3000 Da or equal to 5000 Da or equal to 50000 Da.

According to an embodiment, a composition in the form of a thermoformed extrudate comprising at least one labdane diterpene (or labdane) and at least one natural or synthetic amino acid as a polymer is therefore provided according to the invention.

As an example, among natural or synthetic amino acids, glycine, lysine, proline, tyrosine, arginine, derivatives thereof and mixtures thereof can be mentioned.

Advantageously, the composition according to the invention in the form of a thermoformed extrudate comprises at least one labdane diterpene (or labdane) and at least one natural or synthetic amino acid as a polymer, in particular a natural or synthetic proteinogenic amino acid as a polymer, for example, asparagine, aspartate or aspartic acid, cysteine, glutamate or glutamic acid, glycine, histidine, isoleucine, leucine, lysine, methionine, phenylalanine, proline, serine, threonine, tryptophane, tyrosine or valine.

Other natural or synthetic amino acids or derivatives of natural or synthetic amino acids can be mentioned, like for example, hydroxyproline, citrulline, ornithine or also taurine.

According to an embodiment, a composition in the form of a thermoformed extrudate comprises at least one labdane diterpene (or labdane) and at least one peptide or a natural or synthetic polypeptide amino acid as a polymer is therefore provided according to the invention.

As an example, among natural or synthetic peptides and polypeptides, aspartame, carnosine, insulin, glutathione, derivatives thereof and mixtures thereof can be mentioned.

According to an embodiment, a composition in the form of a thermoformed extrudate comprising at least one labdane diterpene (or labdane) and at least one natural or synthetic oligosaccharide as a polymer is therefore provided according to the invention.

As an example, among oligosaccharides, cyclodextrins, raffinose, rhamnose, saccharose, stachyose, verbascose, trehalose, lactose, lactulose, maltose, derivatives thereof and mixtures thereof can be mentioned.

According to an embodiment, a composition in the form of a thermoformed extrudate comprises at least one labdane diterpene (or labdane) and at least one natural or synthetic cyclodextrin as a polymer can also be provided according to the invention.

As an example, among cyclodextrins, α-cyclodextrin, β-cyclodextrin, derivatives thereof and mixtures thereof can be mentioned.

Advantageously, according to the invention said natural or synthetic non-cellulose polysaccharides are selected from the group made up of dextrins, alginates, hyaluronates, carrageenans, starches, derivatives thereof and mixtures thereof.

According to an embodiment, a composition in the form of a thermoformed extrudate comprises at least one labdane diterpene (or labdane) and at least one natural or synthetic non-cellulose polysaccharide as a polymer is therefore provided according to the invention.

According to an embodiment, a composition in the form of a thermoformed extrudate comprises at least one labdane diterpene (or labdane) and at least one natural or synthetic dextrin as a polymer is provided according to the invention.

According to an embodiment, a composition in the form of a thermoformed extrudate comprises at least one labdane diterpene (or labdane) and at least one natural or synthetic alginate as a polymer is provided according to the invention.

As an example, among alginates, alginic acid, sodium alginate, calcium alginate, derivatives thereof and mixtures thereof can be mentioned.

According to an embodiment, a composition in the form of a thermoformed extrudate comprises at least one labdane diterpene (or labdane) and at least one natural or synthetic hyaluronate as a polymer is also provided according to the invention.

As an example, among hyaluronates, hyaluronic acid, sodium hyaluronate, derivatives thereof and mixtures thereof can be mentioned.

According to an embodiment, a composition in the form of a thermoformed extrudate comprises at least one labdane diterpene (or labdane) and at least one natural or synthetic carrageenan as a polymer is also provided according to the invention.

As an example, among carrageenans, κ-carrageenan, I-carrageenan, λ-carrageenan, derivatives thereof and mixtures thereof can be mentioned.

By the term “natural amino acid”, this means, in the sense of the present invention, any amino acid naturally present in the living world, in particular a plant or animal amino acid.

By the term “synthetic amino acid”, this means, in the sense of the present invention, any amino acid forming the subject of a human intervention, in particular an amino acid obtained at the start of a chemical or biochemical or biotechnological process.

By the term “natural peptide or polypeptide”, this means, in the sense of the present invention, any peptide or polypeptide naturally present in the living world, in particular a plant or animal peptide or polypeptide.

By the term “synthetic peptide or polypeptide”, this means, in the sense of the present invention, any peptide or polypeptide forming the subject of a human intervention, in particular a peptide or a polypeptide obtained at the start of a chemical or biochemical or biotechnological process.

By the term “natural protein”, this means, in the sense of the present invention, any protein naturally present in the living world, in particular a plant or animal protein.

By the term “synthetic protein”, this means, in the sense of the present invention, any protein forming the subject of a human intervention, in particular a protein obtained at the start of a chemical or biochemical or biotechnological process.

By the term “natural oligosaccharide”, this means, in the sense of the present invention, any oligosaccharide naturally present in the living world, in particular a plant or animal oligosaccharide.

By the term “synthetic oligosaccharide”, this means, in the sense of the present invention, any oligosaccharide forming the subject of a human intervention, in particular any oligosaccharide obtained at the start of a chemical or biochemical or biotechnological process.

By the term “natural non-cellulose polysaccharide”, this means, in the sense of the present invention, any non-cellulose polysaccharide naturally present in the living world, in particular a plant or animal non-cellulose polysaccharide.

By the term “synthetic non-cellulose polysaccharide”, this means, in the sense of the present invention, any non-cellulose polysaccharide forming the subject of a human intervention, in particular any non-cellulose polysaccharide obtained at the start of a chemical or biochemical or biotechnological process.

Advantageously, according to the invention, said thermoformed extrudate comprises a thermoformed mixture of at least one labdane diterpene (or labdane) and of at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

Alternatively, according to the invention, said thermoformed extrudate is constituted of a thermoformed mixture of at least one labdane diterpene (or labdane) and of at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

A thermoformed extrudate obtained by hot melt thermoforming, in particular obtained by hot melt extrusion is therefore provided according to the invention, said thermoformed extrudate comprising a thermoformed mixture of at least one labdane diterpene (or labdane) and of at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

By the term “thermoformed mixture”, this means, in the sense of the present invention, a mixture which exits from equipment, in particular which exits from an extruder, wherein it has undergone a transformation (change of state of the material) by the effect of heat, possibly by the combined effect of heat and shearing forces of a worm screw. Such a transformation by the effect of heat, possibly by the combined effect of heat and shearing forces of a worm screw, can be obtained with the hot melt extrusion (HME) technique.

A thermoformed mixture according to the invention can therefore be obtained according to the hot melt extrusion technique, which makes it possible to achieve the molecular dispersion of an agent (active substance) within a polymeric matrix (within a polymer) to form solid dispersions, the agent and/or the polymer implemented undergoing a change of state of the material. The agent and/or the polymer therefore passes from a first state of the material to a second state of the material, which second state of the material implying that said agent and/or said polymer is presented at least partially in amorphous form.

In particular, a thermoformed mixture according to the invention is a mixture wherein said at least one labdane diterpene (or labdane) and/or said at least one polymer has/have been melted to lead to a solid dispersion consisting of a glassy structure comprising a molecular mixture of said at least one labdane diterpene (or labdane) and of said at least one polymer, such that said at least one labdane diterpene (or labdane) is dispersed within said at least one polymer.

According to the invention, said at least one labdane diterpene (or labdane) comprises a first amorphous phase. More specifically, according to the invention, said at least one labdane diterpene (or labdane), for example andrographolide, comprises at least one first amorphous phase.

Advantageously, according to the invention, said at least one labdane diterpene (or labdane) mainly comprises at least one first amorphous phase. More specifically, according to the invention, said at least one labdane diterpene (or labdane), for example andrographolide, mainly comprises at least one first amorphous phase.

By the term “mainly at least one first amorphous phase”, this means, in the sense of the present invention, that said at least one labdane diterpene (or labdane) comprises between 51 and 100% by mass of an amorphous phase and between 0 and 49% of a crystalline phase.

Preferably, according to the invention, said at least one labdane diterpene (or labdane) comprises between 51 and 100% by mass of an amorphous phase and between and 49% of a crystalline phase. More specifically, according to the invention, said at least one labdane diterpene (or labdane), for example andrographolide, comprises between 51 and 100% by mass of an amorphous phase and between 0 and 49% of a crystalline phase.

According to the invention, preferably, said at least one labdane diterpene (or labdane), for example andrographolide, comprises at least one first amorphous phase and possibly a second crystalline phase.

By the term “comprises at least one first amorphous phase and possibly a second crystalline phase”, this means, in the sense of the present invention, that said at least one labdane diterpene (or labdane), for example andrographolide, can either comprise 100% by mass of an amorphous phase, or that it can comprise simultaneously a first amorphous phase and a second crystalline phase, the sum of the percentages by mass of the first and second phases being, in this case, equal to 100. In other words, the composition according to the invention, can comprise said at least one labdane diterpene (or labdane), for example andrographolide, (1) totally in amorphous form, or (2) partially in amorphous form (phase) and partially in crystalline form (phase).

It is noted that a phase is called amorphous when the atoms constituting it do not respect any order at medium- and long-distance, which distinguishes it from a so-called crystalline phase.

The composition according to the invention is therefore presented preferably in the form of a thermoformed extrudate, wherein said at least one labdane diterpene (or labdane) (principal agent), for example andrographolide, comprises at least one first amorphous phase and possibly a second crystalline phase, which phase(s) is/are dispersed within at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

Advantageously, the composition according to the invention further comprises at least one plasticising agent. The addition of a plasticising agent in a composition according to the invention makes it possible to obtain a composition according to the invention through a manufacturing method where temperatures less than the melting points of said at least one labdane diterpene (or labdane) (principal agent), for example andrographolide, and of the polymer can be used in order to guarantee, all the same, a melting of these two compounds and the dispersion/distribution of said at least one labdane diterpene (or labdane) (principal agent), for example andrographolide, within the polymer.

Preferably, said at least one plasticising agent is selected from the group made up of polyols, lipides, lecithins, sucrose esters, water, triethyl citrate, glycol polyethylene, glycerol, dibutyl sebacate, butyl stearate, glycerol monostearate, diethyl phthalate, derivatives thereof and mixtures thereof.

According to the invention, the preferred plasticising agents are glycerol, water, glycol polyethylene and triethyl citrate.

Advantageously, according to the invention, said at least one plasticising agent is present at a rate of 5% to 30% by weight with respect to the total weight of the composition, preferably at a rate of 10% to 25% by weight with respect to the total weight of the composition, preferably at a rate of 15% to 20% by weight with respect to the total weight of the composition.

Advantageously, according to the invention, said at least one plasticising agent is present at a rate of 5% by weight with respect to the total weight of the composition, or at a rate of 10% by weight with respect to the total weight of the composition, at a rate of 15% by weight with respect to the total weight of the composition, at a rate of 20% by weight with respect to the total weight of the composition, at a rate of 25% by weight with respect to the total weight of the composition, at a rate of 30% by weight with respect to the total weight of the composition.

Preferably, the composition according to the invention further comprises at least one additive selected from the group made up of lubricating agents, surfactant agents, antioxidising agents, chelating agents, derivatives thereof and mixtures thereof.

As an example, the following compounds can be used, by themselves or in a mixture, as lubricating agents in a composition according to the invention: glycerol dibehenate, talc, silica, stearic acid, boric acid, waxes, sodium oleate, sodium acetate, magnesium stearate, calcium stearate, sodium stearate, sodium benzoate, sodium laurylsulfate, glycerol distearate, glycerol palmitostearate, microcrystalline cellulose or also polyoxyl-8-glycerides.

As an example, the following compounds can be used, by themselves or in a mixture, as surfactant agents in a composition according to the invention: Pluronic®, Span®, Cremophor®, polysorbates (Tween®, etc.), vitamin E TPGS and sodium ducosate.

As an example, the following compounds can be used, by themselves or in a mixture, as antioxidising agents and/or chelating agents in a composition according to the invention: butylated hydroxytoluene, butylated hydroxyanisiole, EDTA, citric acid and vitamin E.

Advantageously, the composition according to the invention further comprises at least one additional compound of the polyphenol type selected from the group made up of phenolic acids, stilbenes, phenolic alcohols, lignans, flavonoids, protoberberine alkaloids (berberine), derivatives thereof and mixtures thereof. In particular, the glycosylated and aglycone forms of polyphenols are considered as an additional principal agent according to the present invention. More specifically, in the sense of the present invention, the term “polyphenol” means both natural origin polyphenols and synthetic polyphenols, but also all polyphenol derivatives.

As an example, in the sense of the present invention, hydrobenzoic acid (gallic acid, tannic acid, etc.) derivatives and hydroxycinnamic acid (curcumin, coumaric acid, caffeic acid, ferulic acid, etc.) derivatives can be cited as phenolic acids.

As an example, in the sense of the present invention, resveratrol, sirtinol, piceatannol or also polydatin can be cited as stilbenes.

As an example, in the sense of the present invention, flavanols (quercetin, myricetin, kaempferol, isorhamnetin, morin, rutin, tiliroside, trihydroxyethylrutin, fisetin, etc.), flavones (apigenin, luteolin, baicalein, chrysin, diosmin, nobiletin, tangeretin, wogonin, aminogenistein, etc.), flavanones (bavachin, 8-isopentenylnaringenin, isoxanthohumol, naringenin, eriodictyol, hesperetin, silybin, taxifolin, etc.), isoflavones (genistein, daidzein, daidzin, formonetin, genistin, neobavaisoflavone, pueranin, etc.), anthocyanidins (cyanidin, pelargonidin, delphinidin, petunidin, malvidin, etc.) and flavanols (catechins, gallocatechin, epigallocatechingallate, etc.) can be cited as flavonoids.

According to the invention, said at least one additional principal agent of the polyphenol type constitutes an efflux pump inhibitor/modulator, including P-gp.

Preferably, the composition according to the invention further comprises at least one inhibitor and/or one modulator of the activity of P-gp.

Preferably, the composition according to the invention is packaged in the form of pellets, flakes, granules, powders, effervescent or not tablets, injectable or not solutions, suspensions, gels, creams or also in any other suitable form enabling an administration to an animal or to a human being.

According to an embodiment, the composition according to the invention further comprises at least one additional natural or synthetic polymer, for example, polyvinyl acetate, polyvinylpyrrolidone (PVP), polyvinylpyrrolidone-co-vinyl acetate, polyethylene-co-vinyl acetate, polyvinyl acid co-methacrylic acetate, oxide polyethylene, polylactide-co-glycolide, polyvinyl alcohol, polycarbophil, polycaprolactone, carnauba wax, ethylene-vinyl copolymer, lecithin, castor oil, hydrogenated soya oil, waxes, isomalt, derivatives thereof and mixtures thereof.

Other forms of embodiment of a composition according to the invention are indicated in the accompanying claims.

The invention also aims for a manufacturing method, in particular method for manufacturing by thermoforming, a composition in the form of a thermoformed extrudate according to the invention, characterised in that it comprises the following steps:
a) a simultaneous or deferred step of supplying, over time, at least one labdane diterpene (or labdane) and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, to supply an extruder,
b) a step of mixing, in said extruder, said at least one labdane diterpene (or labdane) and said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, to form a mixture, and
c) a step of hot melt extruding said mixture obtained in step b) in said extruder to obtain a thermoformed extrudate, in particular to obtain a thermoformed extrudate consisting of a solid dispersion, more specifically to obtain a thermoformed extrudate consisting of a solid dispersion consisting of a glassy structure comprising a molecular mixture of said at least one labdane diterpene (or labdane) and of said at least one polymer.

It has been shown, in the scope of the present invention, that said at least one labdane diterpene (or labdane), for example andrographolide, is not degraded even during the method for manufacturing by thermoforming the composition in the form of a thermoformed extrudate which however involves the submission of said at least one labdane diterpene (or labdane), for example andrographolide, at high temperatures (HME). Moreover, it has also been highlighted that said at least one labdane diterpene (or labdane), for example andrographolide, in a composition in the form of a thermoformed extrudate according to the invention is homogeneously distributed there.

More specifically, the hot melt extrusion (HME) carried out according to the method for manufacturing by thermoforming according to the invention leads to a melting of the principal agent (said at least one labdane diterpene) and/or said at least one polymer at a temperature greater than or equal to their melting point.

However, according to certain embodiments of a composition according to the invention, this melting of the principal agent and of the polymer can lead to a temperature less than their melting point. This is, for example, the case if the composition according to the invention comprises a plasticising agent or if the principal agent itself has plasticising properties. Such a melting of the principal agent and/or of the polymer leads to a solid dispersion wherein the principal agent (said diterpenic lactone) preferably comprising at least one first amorphous phase and possibly a second crystalline phase is homogeneously dispersed/distributed within said at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, to form a mixture.

Advantageously, according to the method according to the invention, the hot melt extrusion step c) is a step according to which said at last one labdane diterpene (or labdane) and/or said at least one polymer undergoes/undergo a change of state of the material, said at least one labdane diterpene (or labdane) and/or said at least one polymer passing from a first state of the material to a second state of the material, which second state of the material implying in particular that said at least one labdane diterpene (or labdane) and/or said at least one polymer is/are presented at least partially in amorphous form.

Advantageously, the method according to the invention comprises a prior step of premixing said at least one labdane diterpene (or labdane) and said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, so as to form a premixture intended to supply the extruder.

Preferably, according to the method according to the invention, said hot melt extrusion step is carried out at an extrusion temperature or thermoforming temperature of between 20 and 180° C., preferably at a temperature of between 40 and 115° C., preferably at a temperature of between 80 and 90° C., preferably at a temperature equal to 115° C., preferably at a temperature equal to 110° C., preferably at a temperature equal to 100° C. Advantageously, according to the method according to the invention, said hot

melt extrusion step is carried out at a rotation speed of an extrusion screw of between 20 and 900 rpm, preferably of between 50 and 300 rpm, preferably of between 100 and 250 rpm, preferably equal to 250 rpm, preferably equal to 200 rpm.

Preferably, according to the method according to the invention, said hot melt extrusion step is carried out at a rotation speed of an extrusion screw of between 400 and 600 rpm.

Preferably, the method according to the invention comprises an additional step of cooling exiting from the extruder.

Advantageously, the method according to the invention comprise an additional step of treating the thermoformed extrudate exiting from the extruder, for example a cutting at the level of a pelletiser and/or a crushing of said thermoformed extrudate.

Other embodiments of the method according to the invention are indicated in the accompanying claims.

The present invention also relates to a composition in the form of a thermoformed extrudate obtained according to the method according to the invention, said composition comprising at least one labdane diterpene (or labdane), for example andrographolide, as principal agent and at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, said at least one labdane diterpene (or labdane), for example andrographolide, preferably comprising at least one first amorphous phase and possibly a second crystalline phase.

In other words, the present invention also relates to a composition in the form of a thermoformed extrudate obtained by hot melt extrusion (HME), said composition comprising at least one labdane diterpene (or labdane), for example andrographolide, as principal agent and at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, said at least one labdane diterpene (or labdane), for example andrographolide, preferably comprising at least one first amorphous phase and possibly a second crystalline phase.

The present invention also relates to a use of a composition according to the invention as a food supplement and/or as a cosmetic product and/or as a medication for human or veterinary use.

In particular, the present invention relates to a composition for use in preventive and/or curative treatment, in human beings and/or in animals, of pathologies in line with the respiratory system (cold, influenza, respiratory infections, etc.), pathologies in line with the immune system, the cardiovascular system (hypotension, vasoconstriction, ventricular hypertrophy, arrythmia, etc.), pathologies in line with the blood system (cholesteraemia, platelet aggregation, etc.), pathologies in line with the premature ageing of cells, pathologies in line with the endocrinal system (hyperglycaemia, hypercholesteraemia, etc.), pathologies in line with the central nervous system, skin diseases, diseases due to the presence of microorganisms and cancers (anti-tumoral, etc.) and in preventive and/or curative treatment, in human beings and/or in animals, of pain and of fever.

More specifically, the present invention relates to a composition for use in preventive and/or curative treatment in human beings and/or in animals, diseases linked to respiratory infections, to the premature ageing of cells, obesity, diabetes, hypercholesteraemia.

Other forms of use of a composition according to the invention are indicated in the accompanying claims.

A composition according to the invention preferably has anti-inflammatory, antimicrobial, antiviral, immunomodulator, antipyretic, analgesic, lipid-lowering, antioxidising, antithrombotic, antitumoral, antidiabetic, hepatoprotective, nephroprotective properties, as well as neuroprotective properties.

In particular, a composition according to the invention has antiviral properties, in particular antiviral properties against viral infections of the respiratory tract, observed for example in case of cold or influenza.

Furthermore, advantageously, a composition according to the invention has properties making it possible to decrease fever, inflammation, pain and/or discomfort in a patient or in an animal.

Other features, details and advantages of the invention will emerge from the examples given below, in a non-limiting manner and by making reference to the accompanying figures.

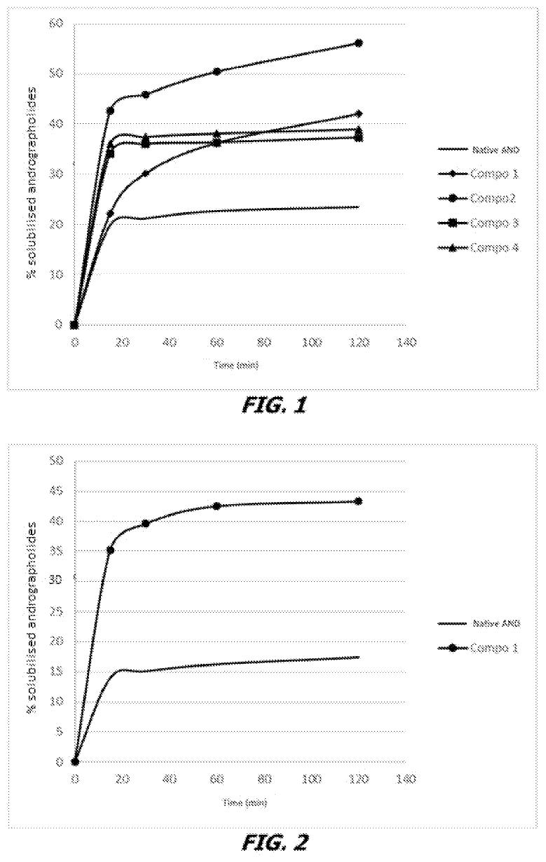

FIG. 1 is a graph illustrating the solubilisation rate of a labdane diterpene (or labdane), in this case, the solubilisation rate of andrographolide over time, at the start of examples of compositions according to the invention.

FIG. 2 is a graph illustrating the solubilisation rate of a labdane diterpene (or labdane), in this case, the solubilisation rate of andrographolide over time, at the start of another example of a composition according to the invention.

FIGS. 3 to 10 are graphs illustrating the solubilisation rate of a labdane diterpene (or labdane), in this case, the solubilisation rate of andrographolide over time, at the start of other examples of compositions according to the invention.

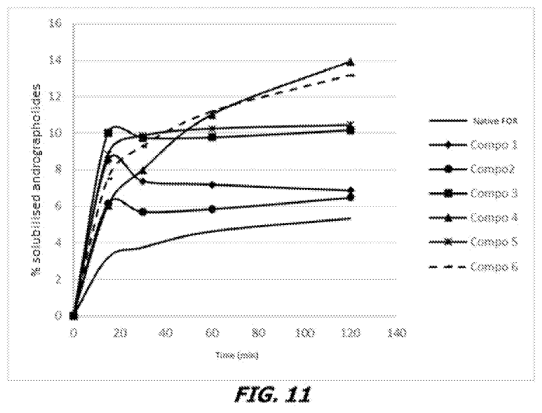

FIG. 11 is a graph illustrating the solubilisation rate of a labdane diterpene (or labdane), in this case, the solubilisation rate of forskolin over time, at the start of other examples of compositions according to the invention.

### EXAMPLES

### Example 1: Method for Manufacturing by Thermoforming a Composition According to the Invention in the Form of a Thermoformed Extrudate

A thermoformed composition according to the invention comprising at least one labdane diterpene (or labdane), such as that forming the subject of example 2 below, has been obtained according to the following method, also forming the subject of the present invention:
a) a step of premixing at least one labdane diterpene (or labdane) in the crystalline state in the form of powder and at least one polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof;
b) a step of supplying said premixture formed in step a) to supply a Process 11 Hygienic TSE Thermo-Fisher®-type extruder;
c) a step of mixing, in said extruder, said premixture to obtain a mixture;
d) a step of thermoforming by hot melt extrusion said mixture obtained in step c) in said extruder to obtain a thermoformed extrudate, the hot melt extrusion step being carried out at a rotation speed of an extrusion screw of 200 rpm and at a temperature of between 20 and 100° C.;
e) a step of cooling, exiting from the extruder, said thermoformed extrudate obtained in step d); and
f) a step of cutting/crushing, at the level of a crusher, the cooled thermoformed extrudate obtained in step e), so as to obtain a homogeneous powder.

The thermoforming temperature at which the hot melt extrusion step is carried out is determined by the type of constituents implemented, in particular according to the type of polymer and/or plasticising agent implemented, which a person skilled in the art is able to determine. Moreover, a person skilled in the art, in particular according to the type of extruder used and according to the general principle of hot melt extrusion (HME), is able to define possible levels of temperature in different zones along the extrusion screw(s) such that a progressive increase of temperature occurs within the material transported by the extrusion screw(s), this in an advance direction of the material within the extruder. Typically, between zones defined along the extrusion screw(s), temperature differences of around 0 to 40° C. are observed. For example, in the scope of the present invention, the compositions tested below have been obtained in a Process 11 Hygienic TSE Thermo-Fisher®-type extruder having 8 temperature zones which are the following in an advance direction of the material developing at a speed of 100 rpm: 20° C.-20° C.-20° C.-60° C.- 80° C.-90° C. and 100° C.

### Example 2: Test of Solubility of Thermoformed Compositions According To The Invention

Thermoformed composition, obtained according to the manufacturing method described in example 1, have been tested in terms of solubility of andrographolides present in an Andrographis paniculata extract standardised at 20% andrographolides. This solubility has been measured over time at the start of thermoformed extrudates obtained according to the invention. The thermoformed extrudates consist of solid dispersions being presented in the form of a homogeneous powder (crushed material).

The solubility test has been carried out with a palette dissolution device at the start of around 1.5 g of thermoformed extrudate, at a temperature of 37° C. under stirring at in 900 ml of an HCl 0.1 N dissolution medium. This solubility test has been carried out according to the recommendations of the pharmacopeia Ph.Eur.9.0 (Recommendations of Dissolution Testing). At determined times, a sample of 1 ml of mixture has been sampled to carry out a solubility test.

In order to carry out the solubility tests, the tested sample has been centrifuged at a speed of 10000 rpm for 10 minutes, then diluted in the HPLC mobile phase before HPLC analysis (Nucleodur 100-5 EC C18 125/4 column (Macherey-Nagel); mobile phase: 40%—A (acetonitrile) and 60% B (Water—0.1% H₃PO₄), flow rate: 0.8 ml/min; loop: 20 μl, t°=40° C.).

The thermoformed compositions according to the invention outlined in Table 1 have been formulated according to the method of the invention and tested in terms of solubility over time according to the principle indicated above. An Andrographis paniculata extract standardised at 20% andrographolides in native crystalline form and in powder form (native AND) has been used as a control. The quantities mentioned in Table 1 are percentages by weight of the compounds implemented (subjected to the method according to the invention) with respect to the total weight of the composition.

TABLE 1

| | Andrographis | | | | |
|---|---|---|---|---|---|
| | paniculata | | | | |
| | extract (20%) | Glycerol | | | |
| | (1) | (2) | Oligosaccharide | Protein | Polysaccharide |
| Compo 1 | 40 | 20 | 40 (3) | | |
| Compo 2 | 40 | 20 | 40 (4) | | |
| Compo 3 | 40 | 15 | | 45 (5) | |
| Compo 4 | 40 | 20 | | | 40 (6) |

(1) Andrographic paniculata extract (20% andrographolides) (Konark Herbals)
(2) Glycerol (Sigma-Aldrich)
(3) β-cyclodextrin (Roquette)
(4) γ-cyclodextrin (Ashland)
(5) Bovine collagen having a molecular weight of 5000 Da (Ingrizo)
(6) Dextrin (Tackidex C760)

The results obtained are presented in FIG. 1. As can be observed, the solubilities measured for the andrographolides at the start of the different compositions in the form of thermoformed extrudates according to the invention (Compo 1, Compo 2, Compo 3 and Compo 4) are clearly greater with respect to the control (native AND).

### Example 3: Solubility Test of Another Thermoformed Composition According To the Invention

Another thermoformed composition, obtained according to the manufacturing method described in example 1, has been tested in terms of solubility of andrographolides present in an Andrographis paniculata extract standardised at 40% andrographolides. The same experimental protocol as that described in example 2 has been implemented.

The thermoformed composition according to the invention outlined in Table 2 has been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 2 are percentages by weight of the compounds implemented (subjected to the protocol according to the invention) with respect to the total weight of the composition.

TABLE 2

| | Andrographis | | |
|---|---|---|---|
| | paniculata | | |
| | (40%) (1) | Glycerol (2) | Oligosaccharide |
| Compo 1 | 30 | 20 | 50 (3) |

(1) Andrographis paniculata extract (40% andrographolides) (Konark Herbals)
(2) Glycerol (Sigma-Aldrich)
(3) β-cyclodextrin (Roquette)

The results obtained are presented in FIG. 2. As can be observed, the solubility measured for the andrographolides at the start of the composition in the form of a thermoformed extrudate according to the invention (Compo 1) is clearly greater with respect to the control (native AND).

### Example 4: Solubility Test of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 3 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 3 are percentages by weight of the compounds implemented (subjected to the method according to the invention) with respect to the total weight of the composition.

TABLE 3

| | Andrographis | | |
|---|---|---|---|
| | paniculata | | |
| | extract (40%) | | |
| | (1) | Glycerol (2) | Polysaccharide |
| Compo 1 | 30 | 15 | 55 (3) |
| Compo 2 | 30 | 15 | 55 (4) |
| Compo 3 | 30 | 15 | 55 (5) |
| Compo 4 | 30 | 15 | 55 (6) |

(1) Andrographis paniculata extract (40% andrographolides) (Konark Herbals)
(2) Glycerol (Sigma-Aldrich)
(3) Cleargum CO01 modified starch (Roquette)
(4) Cleargum CB95 modified starch (Roquette)
(5) Cleargum MG85 modified starch (Roquette)
(6) Non-modified maize starch (Maïzena ®)

The results obtained are presented in FIG. 3. As can be observed, the solubility measured for the andrographolides at the start of the compositions in the form of thermoformed extrudates according to the invention (Compo 1, Compo 2, Compo 3 and Compo 4) are greater with respect to the control (native AND).

### Example 5: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 4 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 4 are percentages by weight of the compounds implemented (subjected to the method according to the invention) with respect to the total weight of the composition.

TABLE 4

| | Andrographis | | | | |
|---|---|---|---|---|---|
| | paniculata | | | | |
| | extract | Glycerol | Oligosaccharide | Oligosaccharide | Polysaccharide |
| | (40%) (1) | (2) | (4) | (5) | (3) |
| Compo1 | 30 | 15 | | | 55 |
| Compo 2 | 30 | 15 | 25 | | 30 |
| Compo 3 | 30 | 15 | | 25 | 30 |
| Compo 4 | 30 | 15 | 55 | | |
| Compo 5 | 30 | 15 | | 55 | |

(1) Andrographis paniculata extract (40% andrographolides) (Konark Herbals)
(2) Glycerol (Sigma-Aldrich)
(3) Cleargum CO01 modified starch (Roquette)
(4) β-cyclodextrin (Roquette)
(5) γ-cyclodextrin (Ashland)

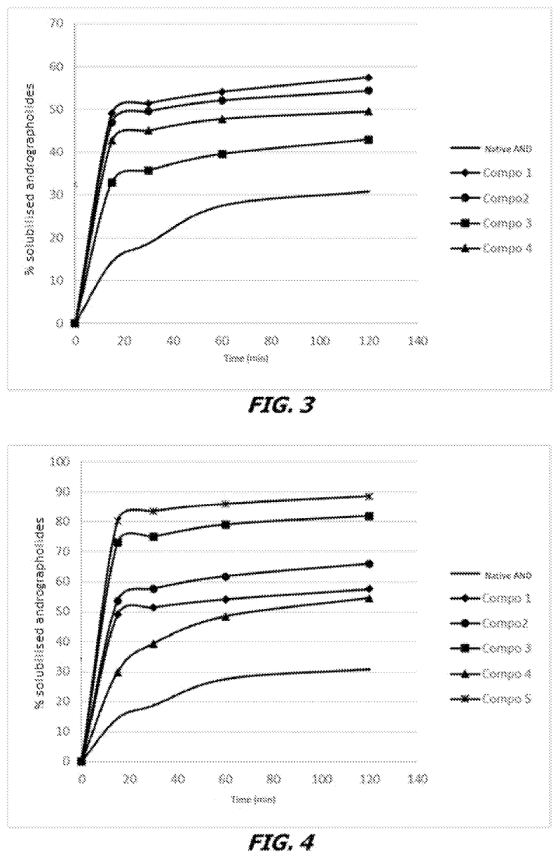

The results obtained are presented in FIG. 4. As can be observed, the solubility measured for the andrographolides at the start of the composition in the form of thermoformed extrudates according to the invention (Compo 1, Compo 2, Compo 3, Compo 4 and Compo 5) are greater with respect to the control (native AND).

### Example 6: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 5 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 5 are percentages by weight of the compounds implemented (subjected to the method according to the invention) with respect to the total weight of the composition.

TABLE 6

| | Andrographis | | | | |
|---|---|---|---|---|---|
| | paniculata | | | | |
| | extract (40%) | Glycerol | Oligosaccharide | Oligosaccharide | Polysaccharide |
| | (1) | (2) | (4) | (5) | (3) |
| Compo 1 | 30 | 15 | | | 55 |
| Compo 2 | 30 | 15 | 25 | | 30 |
| Compo 3 | 30 | 15 | | 25 | 30 |
| Compo 4 | 30 | 15 | 55 | | |
| Compo 5 | 30 | 15 | | 55 | |

(1) Andrographis paniculata extract (40% andrographolides) (Konark Herbals)
(2) Glycerol (Sigma-Aldrich)
(3) Cleargum CB95 modified starch (Roquette)
(4) β-cyclodextrin (Roquette)
(5) γ-cyclodextrin (Ashland)

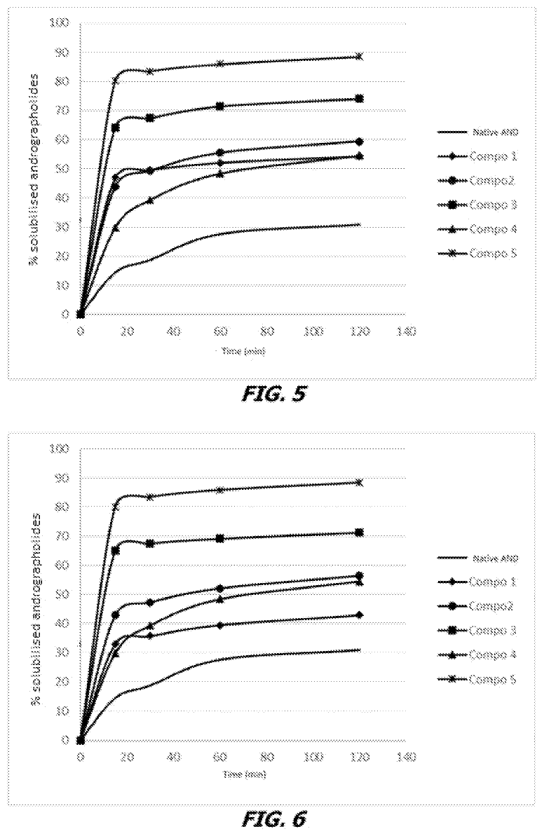

The results obtained are presented in FIG. 5. As can be observed, the solubility measured for the andrographolides at the start of the composition in the form of thermoformed extrudates according to the invention (Compo 1, Compo 2, Compo 3, Compo 4 and Compo 5) are greater with respect to the control (native AND).

### Example 7: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 4 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 6 are percentages by weight of the compounds implemented (subjected to the method according to the invention) with respect to the total weight of the composition.

TABLE 6

| | Andrographis | | | | |
|---|---|---|---|---|---|
| | paniculata | | | | |
| | extract (40%) | Glycerol | Oligosaccharide | Oligosaccharide | Polysaccharide |
| | (1) | (2) | (4) | (5) | (3) |
| Compo 1 | 30 | 15 | | | 55 |
| Compo 2 | 30 | 15 | 25 | | 30 |
| Compo 3 | 30 | 15 | | 25 | 30 |
| Compo 4 | 30 | 15 | 55 | | |
| Compo 5 | 30 | 15 | | 55 | |

(1) Andrographis paniculata extract (40% andrographolides) (Konark Herbals)
(2) Glycerol (Sigma-Aldrich)
(3) Cleargum MG85 modified starch (Roquette)
(4) β-cyclodextrin (Roquette)
(5) γ-cyclodextrin (Ashland)

The results obtained are presented in FIG. 6. As can be observed, the solubility measured for the andrographolides at the start of the composition in the form of thermoformed extrudates according to the invention (Compo 1, Compo 2, Compo 3, Compo 4 and Compo 5) are greater with respect to the control (native AND).

### Example 8: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 7 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 7 are percentages by weight of the compounds implemented (subjected to the method according to the invention) with respect to the total weight of the composition.

TABLE 7

| | Andrographis | | | | |
|---|---|---|---|---|---|
| | paniculata | | | | |
| | extract (40%) | Glycerol | Oligosaccharide | Oligosaccharide | Polysaccharide |
| | (1) | (2) | (4) | (5) | (3) |
| Compo 1 | 30 | 15 | | | 55 |
| Compo 2 | 30 | 15 | 25 | | 30 |
| Compo 3 | 30 | 15 | | 25 | 30 |
| Compo 4 | 30 | 15 | 55 | | |
| Compo 5 | 30 | 15 | | 55 | |

(1) Andrographis paniculata extract (40% andrographolides) (Konark Herbals)
(2) Glycerol (Sigma-Aldrich)
(3) Non-modified maize starch (Maïzena ®)
(4) β-cyclodextrin (Roquette)
(5) γ-cyclodextrin (Ashland)

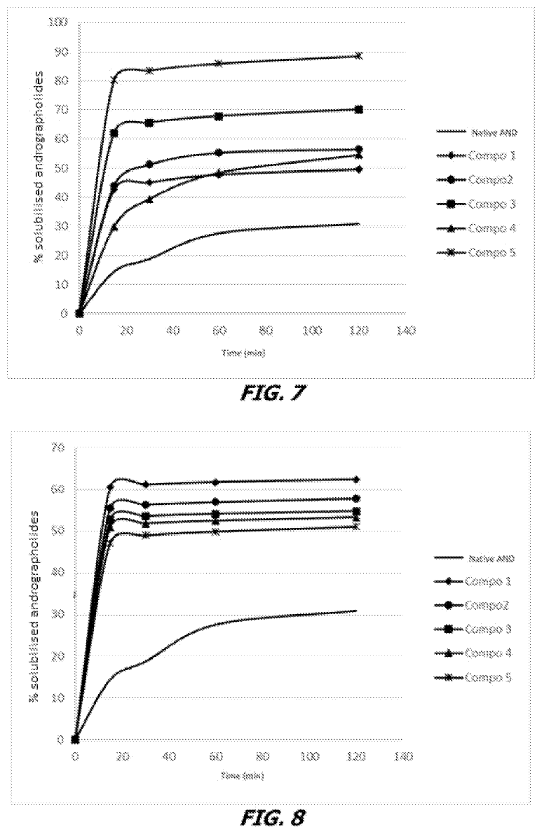

The results obtained are presented in FIG. 7. As can be observed, the solubility measured for the andrographolides at the start of the composition in the form of thermoformed extrudates according to the invention (Compo 1, Compo 2, Compo 3, Compo 4 and Compo 5) are greater with respect to the control (native AND).

### Example 9: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 8 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 8 are percentages by weight of the compounds implemented (subjected to the method according to the invention) with respect to the total weight of the composition.

TABLE 8

| | Andrographis | | |
|---|---|---|---|
| | paniculata | | |
| | extract (40%) | | |
| | (1) | Plasticiser | Protein (2) |
| Compo 1 | 30 | 15 (3) | 55 |
| Compo 2 | 30 | 15 (4) | 55 |
| Compo 3 | 30 | 15 (5) | 55 |
| Compo 4 | 30 | 15 (6) | 55 |
| Compo 5 | 30 | 15 (7) | 55 |

(1) Andrographis paniculata extract (40% andrographolides) (Konark Herbals)
(2) Bovine collagen having a molecular weight of 5000 Da (Ingrizo)
(3) Glycerol (Sigma-Aldrich)
(4) 75% glycerol (Sigma-Aldrich) + 25% water
(5) 50% glycerol (Sigma-Aldrich) + 50% water
(6) 25% glycerol (Sigma-Aldrich) + 75% water
(7) Water

The results obtained are presented in FIG. 8. As can be observed, the solubility measured for the andrographolides at the start of the composition in the form of thermoformed extrudates according to the invention (Compo 1, Compo 2, Compo 3, Compo 4 and Compo 5) are greater with respect to the control (native AND).

### Example 10: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 9 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 9 are percentages by weight of the compounds implemented (subjected to the method according to the invention) with respect to the total weight of the composition.

TABLE 9

| | Andrographis | | |
|---|---|---|---|
| | paniculata | | |
| | extract (40%) | Glycerol | |
| | (1) | (2) | Protein (3) |
| Compo 1 | 20 | 15 (3) | 65 |
| Compo 2 | 25 | 15 (4) | 60 |
| Compo 3 | 30 | 15 (5) | 55 |

(1) Andrographis paniculata extract (40% andrographolides) (Konark Herbals)
(2) Glycerol (Sigma-Aldrich)
(3) Fish collagen having a molecular weight of 2000 Da (Ingrizo)

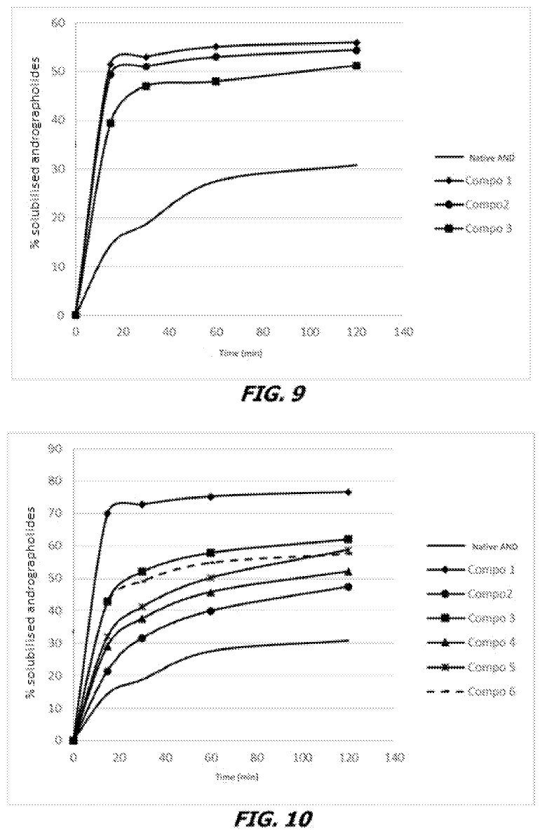

The results obtained are presented in FIG. 9. As can be observed, the solubility measured for the andrographolides at the start of the composition in the form of thermoformed extrudates according to the invention (Compo 1, Compo 2, and Compo 3) are greater with respect to the control (native AND).

### Example 11: Test of Solubility of Other Thermoformed Compositions According to the Invention

Other thermoformed compositions, obtained according to the manufacturing method described in example 1, have been tested in terms of solubility of the andrographolides present in an Andrographis paniculata extract standardised at 40% andrographolides. The same experimental protocol as that described in example 2 has been implemented.

The thermoformed compositions according to the invention outlined in Table 10 have been formulated according to the method of the invention and tested in terms of solubility over time according to the principle indicated above. An Andrographis paniculata extract standardised at 40% andrographolides in native crystalline form and in powder form (native AND) has been used as a control. The quantities mentioned in Table 10 are percentages by weight of the compounds implemented (subjected to the method according to the invention) with respect to the total weight of the composition.

TABLE 10

| | Andrographis | | | | |
|---|---|---|---|---|---|
| | paniculata | | | | |
| | extract (40%) | | | Protein/amino | |
| | (1) | Plasticiser | Oligosaccharide | acid | Polysaccharide |
| Compo 1 | 30 | 15 (2) | 25 (4) | | 30 (9) |
| Compo 2 | 30 | 10 (2) | | 60 (7) | |
| Compo 3 | 30 | 10 (2) | 60 (5) | | |
| Compo 4 | 30 | 10 (3) | | 60 (8) | |
| Compo 5 | 30 | 10 (3) | 60 (6) | | |
| Compo 6 | 30 | 10 (3) | | | 60 (9) |

(1) Andrographis paniculata extract (40% andrographolides) (Konark Herbals)
(2) Glycerol (Sigma-Aldrich)
(3) Glycol polyethylene 400 (ChemLab)
(4) γ-cyclodextrin (Ashland)
(5) Saccharose (Comptoir sucrier)
(6) β-cyclodextrin (Roquette)
(7) Glycine (Freemen)
(8) Bovine collagen having a molecular weight of 5000 Da (Ingrizo)
(9) Cleargum CO03 modified starch (Roquette)

The results obtained are presented in FIG. 10. As can be observed, the solubility measured for the andrographolides at the start of the composition in the form of thermoformed extrudates according to the invention (Compo 1, Compo 2, Compo 3, Compo 4, Compo 5 and Compo 6) are greater with respect to the control (native AND).

### Example 12: Test of Solubility of Other Thermoformed Compositions According to the Invention

Other thermoformed compositions, obtained according to the manufacturing method described in example 1, have been tested in terms of solubility of the forskolin present in a Coleus forskohlii extract standardised at 20% forskolin. This solubility has been measured over time at the start of the thermoformed extrudates according to the invention. The thermoformed extrudates consist of solid dispersions being presented in the form of a homogeneous powder (crushed material). The solubility test has been carried out with a palette dissolution device at the

start of around 1.5 g of thermoformed extrudate, at a temperature of 37° C. under stirring at in 900 ml of an HCl 0.1 N dissolution medium. This solubility test has been carried out according to the recommendation of the pharmacopeia Ph.Eur.9.0 (Recommendations on Dissolution Testing). At determined times, a sample of 1 ml of mixture has been sampled to carry out a solubility test.

In order to carry out solubility tests, the tested sample has been filtered before HPLC analysis: Column (Pursuit 5-C18-4.6*250 mm, 5 μm, Agilent technologies); Mobile phase: 60%-(Acetonitrile) and 40%-B (Water); flow rate: 1 ml/min; λ=210 nm; loop 10 μl; T°=30° C. The thermoformed compositions according to the invention outlined in Table 11

have been formulated according to the method of the invention and tested in terms of solubility over time according to the principle indicated above. A Coleus forskohlii extract standardised at 20% forskolin in native crystalline form and in powder form (native FOR) has been used as a control. The quantities mentioned in Table 11 are percentages by weight of the compounds implemented (subjected to the method according to the invention) with respect to the total weight of the composition.

TABLE 11

| | Coleus | | | | |
|---|---|---|---|---|---|
| | forskohlii | | | | |
| | extract | Plasticiser | | Protein/amino | |
| | (20%) (1) | (2) | Oligosaccharide | acid | Polysaccharide |
| Compo 1 | 30 | 10 | | 60 (3) | |
| Compo 2 | 30 | 10 | 60 (4) | | |
| Compo 3 | 30 | 10 | | | 60 (5) |
| Compo 4 | 30 | 10 | | 60 (6) | |
| Compo 5 | 30 | 10 | | 60 (7) | |
| Compo 6 | 30 | 10 | | 61 (8) | |

(1) Coleus forskohlii extract (20% forskolin) (Vidya Herbs)
(2) Glycerol (Sigma-Aldrich)
(3) Bovine collagen having a molecular weight of 5000 Da (Ingrizo)
(4) β-cyclodextrin (Roquette)
(5) Cleargum CO03 modified starch (Roquette)
(6) Glycine (Freemen)
(7) Proline (Freemen)
(8) Arginine (Freemen)

The results obtained are presented in FIG. 11. As can be observed, the solubility measured for the forskolin at the start of the composition in the form of thermoformed extrudates according to the invention (Compo 1, Compo 2, Compo 3, Compo 4, Compo 5 and Compo 6) are greater with respect to the control (native FOR).

The present invention has been described in relation to specific embodiments, which have a purely illustrative value and must not be considered as limiting. Generally, it will seem clear for a person skilled in the art that the present invention is not limited to the examples illustrated and/or described above.

The use of the verbs “comprise”, “include”, “have”, or any other variant, as well as their conjugations, cannot in any way exclude the present of elements other than those mentioned.

The use of the indefinite article “a”, “an”, or the definite article “the”, to introduce an element does not exclude the present of a plurality of these elements.

1. Composition in the form of a thermoformed extrudate comprising at least one labdane diterpene and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof.

2. Composition according to claim 1, characterised in that said thermoformed extrudate consists of a solid dispersion.

3. Composition according to claim 2, characterised in that said solid dispersion consists of a glassy structure comprising a molecular mixture of said at least one labdane diterpene and of said at least one polymer.

4. Composition according to any one of the preceding claims, characterised in that said at least one labdane diterpene mainly comprises at least one first amorphous phase.

5. Composition according to claim 4, characterised in that said at least one labdane diterpene comprises between 51 and 100% by mass of an amorphous phase and between 0 and 49% of a crystalline phase.

6. Composition according to any one of the preceding claims, characterised in that said labdane diterpene is selected from the group made up of andrographolide, neoandrographolide, deoxyandrographolide, dehydroandrographolide, isoandrographolide, forskolin, marrubiin, solicanolide, sclareol, marrubenol, galanolactone, medigenine, coronarines, andalusol, hispanolone, copalic acid, hedychenone, lagopsines, macranthine A, labdanediol, serralabdanes, leopersine, cis-communic acid, derivatives thereof and mixtures thereof.

7. Composition according to any one of the preceding claims, characterised in that said natural or synthetic amino acids are selected from the group made up of asparagine, aspartate or aspartic acid, cysteine, glutamate or glutamic acid, glycine, histidine, isoleucine, leucine, lysine, methionine, phenylalanine, proline, serine, threonine, tryptophane, tyrosine, valine, arginine, derivatives thereof and mixtures thereof.

8. Composition according to any one of the preceding claims, characterised in that said peptides and said polypeptides are selected from the group made up of aspartame, carnosine, derivatives thereof and mixtures thereof.

9. Composition according to any one of the preceding claims, characterised in that said natural or synthetic proteins are selected from the group made up of glycoproteins, collagens and/or collagen hydrolysates, plant proteins, animal proteins, derivatives thereof and mixtures thereof.

10. Composition according to claim 9, characterised in that said collagens and/or said collagen hydrolysates have a molecular weight of between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

11. Composition according to any one of the preceding claims, characterised in that said natural or synthetic oligosaccharides are selected from the group made up of cyclodextrins, raffinose, rhamnose, stachyose, verbascose, trehalose, lactose, lactulose, maltose, derivatives thereof and mixtures thereof.

12. Composition according to any one of the preceding claims, characterised in that said natural or synthetic non-cellulose polysaccharides are selected from the group made up of dextrins, alginates, hyaluronates, carrageenans, starches, derivatives thereof and mixtures thereof.

13. Composition according to any one of the preceding claims, characterised in that it further comprises at least one plasticising agent.

14. Composition according to claim 13, characterised in that said at least one plasticising agent is selected from the group made up of polyols, lipides, lecithins, sucrose esters, water, triethyl citrate, glycol polyethylene, glycerol, dibutyl sebacate, butyl stearate, glycerol monostearate, diethyl phthalate, derivatives thereof and mixtures thereof.

15. Composition according to any one of the preceding claims, characterised in that it further comprises at least one additive selected from the group made up of lubricating agents, surfactant agents, antioxidising agents, chelating agents, derivatives thereof and mixtures thereof.

16. Composition according to any one of the preceding claims, characterised in that it further comprises at least one first additional compound of the polyphenol type selected from the group made up of phenolic acids, stilbenes, phenolic alcohols, lignans, flavonoids, derivatives thereof and mixtures thereof.

17. Composition according to any one of the preceding claims, characterised in that it is packaged in the form of pellets, flakes, granules, powders, effervescent or not tablets, injectable or not solutions, suspensions, gels, creams or also in any other suitable form enabling an administration to an animal or to a human being.

18. Manufacturing method, in particular method for manufacturing by thermoforming, a composition in the form of a thermoformed extrudate according to any one of claims 1 to 17, characterised in that it comprises the following steps:
a) a simultaneous or deferred step of supplying, over time, at least one labdane diterpene (or labdane) and at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, to supply an extruder,
b) a step of mixing, in said extruder, said at least one labdane diterpene (or labdane) and said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, to form a mixture, and
c) a step of hot melt extruding said mixture obtained in step b) in said extruder to obtain a thermoformed extrudate, in particular to obtain a thermoformed extrudate consisting of a solid dispersion, more specifically to obtain a thermoformed extrudate consisting of a solid dispersion consisting of a glassy structure comprising a molecular mixture of said at least one labdane diterpene (or labdane) and of said at least one polymer.

19. Method according to claim 18, characterised in that it comprises a prior step of premixing said at least one labdane diterpene and said at least one natural or synthetic polymer selected from the group made up of amino acids, natural or synthetic peptides and polypeptides, natural or synthetic proteins, natural or synthetic oligosaccharides, natural or synthetic non-cellulose polysaccharides, derivatives thereof and mixtures thereof, so as to form a premixture intended to supply the extruder.

20. Method according to claim 18 or 19, characterised in that said hot melt extrusion step is carried out at an extrusion temperature of between 20 and 180° C., preferably at a temperature of between 40 and 115° C., preferably at a temperature of between 80 and 90° C., preferably at a temperature equal to 115° C., preferably at a temperature equal to 110° C., preferably at a temperature equal to 100° C.

21. Method according to any one of claims 18 to 20, characterised in that said hot melt extrusion step is carried out at a rotation speed of an extrusion screw of between 20 and 900 rpm, preferably of between 50 and 300 rpm, preferably of between 100 and 250 rpm, preferably equal to 250 rpm, preferably equal to 200 rpm.

22. Method according to any one of claims 18 to 21, characterised in that it comprises an additional step of cooling exiting from the extruder.

23. Method according to any one of claims 18 to 22, characterised in that it comprises an additional step of treating the thermoformed extrudate exiting from the extruder, for example a cutting at the level of a pelletiser and/or a crushing of said thermoformed extrudate.

24. Composition in the form of a thermoformed extrudate according to any one of claims 1 to 17, for use in preventive and/or curative treatment, in human beings and/or in animals, of pathologies in line with the respiratory system (cold, influenza, respiratory infections, etc.), pathologies in line with the immune system, the cardiovascular system (hypotension, vasoconstriction, ventricular hypertrophy, arrythmia, etc.), pathologies in line with the blood system (cholesteraemia, platelet aggregation, etc.), pathologies in line with the premature ageing of cells, pathologies in line with the endocrinal system (hyperglycaemia, hypercholesteraemia, etc.), pathologies in line with the central nervous system, skin diseases, diseases due to the presence of microorganisms and cancers (anti-tumoral, etc.) and in preventive and/or curative treatment, in human beings and/or in animals, of pain and of fever.

25. Composition in the form of a thermoformed extrudate obtained according to the method according to any one of claims 18 to 23, said composition comprising at least one diterpenic lactone and at least one natural or synthetic polymer selected from the group made up of natural or synthetic proteins, natural or synthetic oligosaccharides, natural or synthetic non-cellulose polysaccharides, derivatives thereof and mixtures thereof.
